(19) United States
(12) Patent Application Publication
(10) Pub. No.: US 2018/0036349 A1
(43) Pub. Date: Feb. 8, 2018

(54) Treatment for Chronic Pain

(71) Applicant: Marv Enterprises, LLC, Hermitage, PA (US)

(72) Inventor: Mitchell S. Felder, Hermitage, PA (US)

(21) Appl. No.: 15/786,865

(22) Filed: Oct. 18, 2017

Related U.S. Application Data
(63) Continuation of application No. 14/376,560, filed on Aug. 4, 2014.
(60) Provisional application No. 61/612,474, filed on Mar. 19, 2012.

(51) Int. Cl. A61K 35/30

(52) U.S. Cl. CPC A61K 35/30

(57) ABSTRACT

A method is described to treat chronic pain syndromes by treating extracorporeally a patient's cerebrospinal fluid (CSF). A patient's CSF is treated extracorporeally using a moiety that targets an antigen in the CSF. This moiety facilitates removal of the CSF antigen. The cleansed CSF is then returned to the patient.

### CROSS-REFERENCE TO RELATED APPLICATIONS

This application claims benefit under 35 U.S.C. § 119(e) of U.S. Patent Application No. 61/612,474, filed Mar. 19, 2012, which is hereby incorporated herein by reference in its entirety.

### FIELD OF THE INVENTION

The present invention relates to a treatment for chronic pain syndromes and, in particular, a treatment for affecting neurotransmitters in the central nervous system that cause chronic pain.

### BACKGROUND OF THE INVENTION

Presently more than 25 million Americans are afflicted with chronic pain syndromes. The understanding and treatment of chronic pain syndrome comprises a unique medical disease model. Symptoms are believed to have an underlying organic pathology related to central nervous system neurotransmitters. The neurotransmitters implicated in the chronic pains syndromes include Substance P, 5-hydroxyindoleacetic acid (5-HIAA), vasoactive intestinal peptide (VIP), calcitonin gene-related peptide (CGRP), phospholipase A2 (PLA2), and the proinflammatory cytokines (PCs): Interleukin (IL)-1, IL-6, IL-10, and tumor necrosis factor-alpha. By applying different treatment modalities, physicians can help to prevent acute pain from becoming chronic pain syndromes. Chronic pain syndromes can be treated differently from acute pain. The appropriate use of medications and treatment modalities is an important component in the multidisciplinary approach to chronic pain syndromes. Treatment of chronic pain syndrome has been most successful when a combination of all available treatment modalities was used. A treatment for stopping and possibly reversing the progression of chronic pain syndromes would be extremely beneficial.

### SUMMARY OF THE INVENTION

The present invention relates to a method of extracorporeal treating a patient's cerebrospinal fluid (CSF). U.S. Ser. No. 13/128870, U.S. Ser. No. 13/128177, and U.S. Ser. No. 13/254855 are hereby incorporated by reference. The treatment includes a plurality of stages comprising removing CSF from a patient, applying an extracorporeal treatment to the CSF, and returning the CSF to the patient.

In the first stage of the treatment, the CSF is removed from the patient. A convenient method for removing CSF includes a standard lumbar puncture. In the second stage, a treatment is applied to the CSF. The treatment can include an antibody directed against targeted antigens. The third stage comprises returning the CSF to the patient and can also include removing the treatment from the CSF. Antibodies can be produced that attack targeted antigen, Substance P.

### BRIEF DESCRIPTION OF THE DRAWINGS

FIG. 1 is a partial cross sectional view of a cylinder and tubing used to deliver a treatment to a bodily fluid.

FIG. 2 is a partial cross sectional view showing additional detail of the cylinder and tubing of FIG. 1.

### DETAILED DESCRIPTION OF THE INVENTION

The method of the present invention comprises treating a patient's CSF extracorporeally with an antibody designed to react with a particular targeted antigen (TA): substance P, 5-hydroxyindoleacetic acid (5-HIAA), vasoactive intestinal peptide (VIP), calcitonin gene-related peptide (CGRP), phospholipase A2 (PLA2), and the proinflammatory cytokines (PCs): interleukin (IL)-1, IL-6, IL-10, and tumor necrosis factor-alpha. The antibody can include a moiety, for example, an albumin moiety, that can complex with the target antigen(s)/TAs: substance P, 5-hydroxyindoleacetic acid (5-HIAA), vasoactive intestinal peptide (VIP), calcitonin gene-related peptide (CGRP), phospholipase A2 (PLA2), and the proinflammatory cytokines (PCs): interleukin (IL)-1, IL-6, IL-10, and tumor necrosis factor-alpha and thereby permit efficacious dialysis of the antibody-antigen complex. Dialysis methods are well-known by those of skill in the art.

In an embodiment of the invention, the antibody comprises an albumin moiety and targets the removal of one or more TAs from the CSF.

The target antigens can include substance P, 5-hydroxyindoleacetic acid (5-HIAA), vasoactive intestinal peptide (VIP), calcitonin gene-related peptide (CGRP), phospholipase A2 (PLA2), and the proinflammatory cytokines (PCs): interleukin (IL)-1, IL-6, IL-10, and tumor necrosis factor-alpha in CSF can be differentiated using standard ELISA methodology. ELISA (enzyme-linked immunosorbant assay) is a biochemical technique which allows for the detection of an antigen in a sample. In ELISA, an antigen is affixed to a surface, and then an antibody is used for binding to the antigen. The antibody is linked to an enzyme which enables a color change in the substrate.

An alternative methodology of the present intervention would use a designer antibody with an attached macromolecular moiety instead of an albumin moiety. The macromolecular moiety, attached to the antibody, would be 1.000 mm to 0.005 mm in diameter. The antibody-macromolecular moiety-targeted antigen complex would then be blocked from reentering the patient's CSF and/or body fluid circulation, by using a series of microscreens which contain openings with a diameter 50.0000% to 99.9999% less than the diameter of the designer antibody-macromolecular moiety. The microscreen opening(s) must have a diameter of at least 25 micrometers in order to allow for the passage and return to circulation of the non-pathological inducing CSF constituents.

In another alternative embodiment, the target antigens/TA, for example, substance P, 5-hydroxyindoleacetic acid (5-HIAA), vasoactive intestinal peptide (VIP), calcitonin gene-related peptide (CGRP), phospholipase A2 (PLA2); and the proinflammatory cytokines (PCs), for example, interleukin (IL)-1, IL-6, IL-10, and tumor necrosis factor-alpha; may be captured by using antibody microarrays that contain antibodies to the targeted antigen(s). The antibody microarrays are composed of millions of identical monoclonal antibodies attached at high density on a surface, such as on a glass or plastic slide. After sufficient extracorporeal exposure of the TAs, substance P, 5-hydroxyindoleacetic acid (5-HIAA), vasoactive intestinal peptide (VIP), calcitonin gene-related peptide (CGRP), phospholipase A2 (PLA2), and the proinflammatory cytokines (PCs): interleukin (IL)-1, IL-6, IL-10, and tumor necrosis factor-alpha, to the antibody microarrays, the antibody microarrays-TA may be disposed of using standard medical practice.

In still another alternative methodology, the intervention comprises removing the targeted antigen (s)/TAs: substance P, 5-hydroxyindoleacetic acid (5-HIAA), vasoactive intestinal peptide (VIP), calcitonin gene-related peptide (CGRP), phospholipase A2 (PLA2), and the proinflammatory cytokines (PCs): interleukin (IL)-1, IL-6, IL-10, and tumor necrosis factor-alpha from that CSF by using a designer antibody containing an iron (Fe) moiety. This will then create an Fe-antibody-antigen complex. The iron-containing complex may then be efficaciously removed using a strong, localized magnetic force field.

The treatment can include the removal of the substance P, 5-hydroxyindoleacetic acid (5-HIAA), vasoactive intestinal peptide (VIP), calcitonin gene-related peptide (CGRP), phospholipase A2 (PLA2), and the proinflammatory cytokines (PCs): interleukin (IL)-1, IL-6, IL-10, and tumor necrosis factor-alpha (targeted antigen: TA) from the CSF. The cleansed CSF can then be returned to the patient, such as, for example by using the same catheter that was originally used in removing the CSF. In one embodiment, the treatment of CSF comprises removing 5-25 mL of CSF from a patient, and applying the treatment to CSF before returning it to the patient. The frequency of such treatments would depend upon the underlying symptomatology and pathology of the patient.

An article for performing the method can comprise at least three stages including a first stage, a second stage and a third stage. The first stage comprises removing CSF from a patient. Removal can occur using any convenient method including, for example, a spinal tap. The second stage treats the CSF. The thirds stage returns the treated CSF to the patient.

The method includes removing CSF from a patient in a first stage, treating the CSF and optionally removing the treatment from the CSF in a second stage, and returning the CSF to the patient in a third stage. The CSF can be removed from the patient using any convenient method, including standard lumbar puncture procedure. The second stage can include sequentially passing the extracorporeal bodily fluid through a treatment chamber and a removal module.

The second stage applies a treatment to the CSF, which can include introducing a designer antibody that joins with an antigen in the CSF to form an antibody-antigen complex. The antibody-antigen complex can be removed from the CSF in the removal module. Optionally, the antibody-antigen complex can be conjugated with a second antibody comprising a moiety that increases the efficacy of removal to form an antibody-moiety-antigen complex.

In the third stage, the purified CSF (CSF with removed TA: substance P, 5-hydroxyindoleacetic acid (5-HIAA), vasoactive intestinal peptide (VIP), calcitonin gene-related peptide (CGRP), phospholipase A2 (PLA2), and the proinflammatory cytokines (PCs): interleukin (IL)-1, IL-6, IL-10, and tumor necrosis factor-alpha) is then returned to the patient.

The article of the invention can include two stages. The first stage includes an inlet for CSF and at least one exterior wall defining a treatment chamber that is fluidly connected to a second stage. The second stage comprises a removal module and an outlet for the CSF. In embodiments, the removal module is selected from a group comprising a mechanical filter, a chemical filter, a dialysis machine, a molecular filter, molecular adsorbant recirculating system (MARS), a plasmapharesis unit, or combinations thereof.

An article for performing the method of the invention comprises a first stage including an inlet for CSF and at least one exterior wall defining a treatment chamber that is fluidly connected to a second stage comprising a removal module and an outlet for the CSF. The treatment chamber can include a delivery tube for introducing a treatment into the treatment chamber. In embodiments, the delivery tube comprises a hollow tube including at least one interior wall defining a plurality of holes through which the treatment can be added to the treatment chamber. The treatment can also be delivered through the hollow tube in counter-current mode with reference to the flow of the extracorporeal CSF. The removal module can be any device capable of removing the antibody-antigen complex. In embodiments, the removal module is selected from a group comprising a mechanical filter, a chemical filter, a dialysis machine, a molecular filter, molecular adsorbant recirculating system (MARS), a plasmapharesis unit, or combinations thereof.

In an example, the first stage of the device applies a treatment of an antibody with an attached albumin moiety that targets the antigen(s): substance P, 5-hydroxyindoleacetic acid (5-HIAA), vasoactive intestinal peptide (VIP), calcitonin gene-related peptide (CGRP), phospholipase A2 (PLA2), and the proinflammatory cytokines (PCs): interleukin (IL)-1, IL-6, IL-10, and tumor necrosis factor-alpha. The second stage includes substantial removal of the treatment from the extracorporeal CSF bodily fluid.

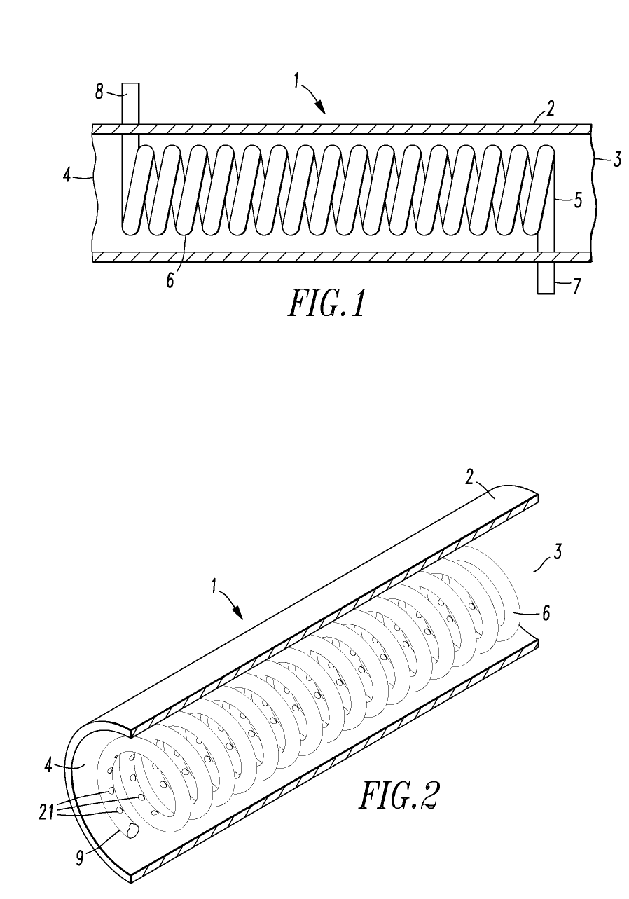

As shown in FIG. 1, the first stage can include an exterior wall 2 defining a treatment chamber 5. The treatment can be applied in the treatment chamber 5. Residence times of the CSF can be altered by changing the dimensions of the treatment chamber or the flow rate of the CSF through the treatment chamber 5. CSF fluid enters the inlet 3, passes through the treatment chamber 5, and exits the outlet 4. In embodiments, the treatment can be applied from a delivery tube 6 located within the treatment chamber 5. An interior wall 9 defines the delivery tube 6. The delivery tube 6 can include at least one lead 7, 8. The lead 7, 8 can deliver the treatment to the treatment chamber 5. Conveniently, the delivery tubes 6 will have a high contact surface area with the CSF. As shown, the delivery tube 6 comprises a helical coil.

With reference to FIG. 2, when the treatment includes the administration of a designer antibody, the delivery tube 6 can be hollow and the interior wall 9 can define a plurality of holes 21. The designer antibodies can be pumped through the delivery tube 6 in order to effect a desired concentration of designer antibodies in the CSF. The designer antibodies can perfuse through the holes 21. The delivery tube 6 can include any suitable material including, for example, metal, plastic, ceramic or combinations thereof. The delivery tube 6 can also be rigid or flexible. In one embodiment, the delivery tube 6 is a metal tube perforated with a plurality of holes. Alternatively, the delivery tube 6 can be plastic.

The antibody with attached albumin moiety and targeting the antigen substance P can be delivered in a concurrent or counter-current mode with reference to the CSF. In counter-current mode, the CSF enters the treatment chamber 5 at the inlet 3. The designer antibody can enter through a first lead 8 near the outlet 4 of the treatment chamber 5. CSF then passes to the outlet 4 and the designer antibodies pass to the second lead 7 near the inlet 3. The removal module of the second stage substantially removes the designer antibodies-antigen molecular compound from the CSF.

The second stage can include a filter, such as a dialysis machine, which is known to one skilled in the art. The second stage can include a molecular filter. For example, molecular adsorbents recirculating system (MARS), which may be compatible and/or synergistic with dialysis equipment. MARS technology can be used to remove small to average sized molecules from the CSF. Artificial liver filtration presently uses this technique.

The methodology can include a plurality of steps for removing the targeted antigen (TA: substance P, 5-hydroxyindoleacetic acid (5-HIAA), vasoactive intestinal peptide (VIP), calcitonin gene-related peptide (CGRP), phospholipase A2 (PLA2), and the proinflammatory cytokines (PCs): interleukin (IL)-1, IL-6, IL-10, and tumor necrosis factor-alpha). A first step can include directing a first antibody against the targeted antigen. A second step can include a second antibody. The second antibody can be conjugated with albumen, or alternatively a moiety that allows for efficacious dialysis. The second antibody or antibody-albumen complex combines with the first antibody forming an antibody-antibody-moiety complex. A third step is then used to remove the complex from the CSF. This removal is enabled by using dialysis and/or MARS. The purified CSF can then be returned to the patient.

In practice, a portion of the purified CSF can be tested to ensure a sufficient portion of the targeted antigen (substance P, 5-hydroxyindoleacetic acid (5-HIAA), vasoactive intestinal peptide (VIP), calcitonin gene-related peptide (CGRP), phospholipase A2 (PLA2), and the proinflammatory cytokines (PCs): interleukin (IL)-1, IL-6, IL-10, and tumor necrosis factor-alpha) has been successfully removed from the CSF. Testing can determine the length of treatment and evaluate the efficacy of the sequential dialysis methodology in removing the targeted antigens. CSF with an unacceptably large concentrations of complex remaining can then be re-filtered before returning the CSF to the patient.

In embodiments, the second stage to remove the antibody-moiety-targeted antigen complex by various techniques including, for example, filtering based on molecular size, protein binding, solubility, chemical reactivity, and combinations thereof. For example, a filter can include a molecular sieve, such as zeolite, or porous membranes that capture complexes comprising molecules above a certain size. Membranes can comprise polyacrylonitrile, polysulfone, polyamides, cellulose, cellulose acetate, polyacrylates, polymethylmethacrylates, and combinations thereof. Increasing the flow rate or dialysate flow rate can increase the rate of removal of the antibody with attached albumin moiety targeting the antigen: substance P, 5-hydroxyindoleacetic acid (5-HIAA), vasoactive intestinal peptide (VIP), calcitonin gene-related peptide (CGRP), phospholipase A2 (PLA2), and the proinflammatory cytokines (PCs): interleukin (IL)-1, IL-6, IL-10, and tumor necrosis factor-alpha.

Additional embodiments can include continuous renal replacement therapy (CRRT), which can remove large quantities of filterable molecules from the extracorporeal CSF. CRRT would be particularly useful for molecular compounds that are not strongly bound to plasma proteins. Categories of CRRT include continuous arteriovenous hemofiltration, continuous venovenous hemofiltration, continuous arteriovenous hemodiafiltration, slow continuous filtration, continuous arteriovenous high-flux hemodialysis, and continuous venovenous high flux hemodialysis.

The sieving coefficient (SC) is the ratio of the molecular concentration in the filtrate to the incoming CSF. A SC close to zero implies that the moiety antibody-targeted antigen complex will not pass through the filter. A filtration rate of 10 mL per min is generally satisfactory. Other methods of increasing the removability of the moiety-antibody-targeted antigen include the use of temporary acidification of the CSF using organic acids to compete with protein binding sites.

Embodiments of the present invention include a method for treating cerebrospinal fluid (CSF) comprising:
a. removing CSF from a patient in a first stage;
b. applying a treatment to the CSF in a second stage; and
c. returning the CSF to the patient in a third stage.

Embodiments of the present invention also include such a method wherein the treatment includes
d. introducing an antibody that joins with an antigen in the CSF to form an antibody-antigen complex; and
e. removing the complex form the CSF.

Embodiments of the present invention also include such a method wherein the targeted antigen is selected from a group consisting of substance P, 5-hydroxyindoleacetic acid (5-HIAA), vasoactive intestinal peptide (VIP), calcitonin gene-related peptide (CGRP), phospholipase A2 (PLA2), and the proinflammatory cytokines (PCs): interleukin (IL)-1, IL-6, IL-10, tumor necrosis factor-alpha, and combinations thereof.

Embodiments of the present invention include a method for treating cerebrospinal fluid (CSF) comprising:
a. removing CSF from a patient in a first stage;
b. applying a treatment to the CSF in a second stage; and
c. returning the CSF to the patient in a third stage wherein the treatment includes
d. introducing a targeted antibody that joins with an antigen in the CSF to form an antibody-antigen complex;
e. conjugating the antibody-antigen complex with a second antibody comprising a moiety that increases efficacy of removal to form an antibody-moiety-antigen complex; and
f. removing the complex from the CSF.

Embodiments of the present invention also include a method for treating cerebrospinal fluid (CSF) wherein the method includes testing the CSF after the treatment and before returning the CSF to the patient in order to determine efficacy of treatment.

Numerous modifications and variations of the present invention are possible. It is, therefore, to be understood that within the scope of the following claims, the invention may be practiced otherwise than as specifically described. While this invention has been described with respect to certain preferred embodiments, different variations, modifications, and additions to the invention will become evident to persons of ordinary skill in the art. All such modifications, variations, and additions are intended to be encompassed within the scope of this patent, which is limited only by the claims appended hereto.

All documents, books, manuals, papers, patents, published patent applications, guides, abstracts and other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method for treating cerebrospinal fluid (CSF) comprising:
a. removing CSF from a patient in a first stage;
b. applying a treatment to the CSF in a second stage; and
c. returning the CSF to the patient in a third stage.

2. The method of claim 1, wherein the treatment includes
a. introducing an antibody that joins with an antigen in the CSF to form an antibody-antigen complex; and
b. removing the complex form the CSF.

3. The method of claim 2, wherein the targeted antigen is selected from a group consisting of substance P, 5-hydroxyindoleacetic acid (5-HIAA), vasoactive intestinal peptide (VIP), calcitonin gene-related peptide (CGRP), phospholipase A2 (PLA2), and the proinflammatory cytokines (PCs): interleukin (IL)-1, IL-6, IL-10, tumor necrosis factor-alpha, and combinations thereof.

4. The method of claim 1, wherein the treatment includes
a. introducing a targeted antibody that joins with an antigen in the CSF to form an antibody-antigen complex;
b. conjugating the antibody-antigen complex with a second antibody comprising a moiety that increases efficacy of removal to form an antibody-moiety-antigen complex; and
c. removing the complex from the CSF.

5. The method of claim 4, wherein the method includes testing the CSF after the treatment and before returning the CSF to the patient in order to determine efficacy of treatment.
